(19) United States
(12) Patent Application Publication
(10) Pub. No.: US 2006/0106466 A1
(43) Pub. Date: May 18, 2006

(54) Process and method for limiting the behavior of an electromechanical device being controlled by one or more remote users

(75) Inventors: Stephen E. Decker, Los Altos, CA (US); Timothy Patrick Keliher, San Jose, CA (US); Brendan Wayne Keliher, San Jose, CA (US)

Correspondence Address: Far Touch Inc., 1622 Marymeade Lane, Los Altos, CA, 94024, US

(73) Assignee: Far Touch Inc.

(21) Appl. No.: 10/990,211

(22) Filed: Nov. 16, 2004

(51) Int. Cl. G05B 15/00; A61F 5/00

(52) U.S. Cl. 700/1; 600/38

(57) ABSTRACT

A process enabling an individual to dynamically refine limits that govern the behavior of an electromechanical device (40) being controlled by a remote operator (10) residing in a different location. The process defines a means by which the control requests created by a remote operator are compared to one or more values, established by the recipient, that define parameters for acceptable behavior or use (63). In the event that the requested action is less than the limit value, the actual request will be released to the electromechanical device (64). If the action exceeds the limit value defined by the recipient, only the limit value will be sent to the device (65). In the case where the requested control value exceeds the limit value set by the recipient, the recipient will be notified such that a particular request exceeded the limitations placed on the device. The limit values can be invisible to the remote operator creating the control requests or, in an alternate embodiment, the information can be provided to the remote operator to help educate the remote user regarding the wishes of the recipient.

### FEDERALLY SPONSORED RESEARCH

Not Applicable. This invention was not made under a government contract. The government owns no interest in this invention.

### SEQUENCE LISTING OR PROGRAM

An example of the code used to perform the limit feature is included as Appendix Item I at the conclusion of this document.

### BACKGROUND

1. Field of Invention

This invention relates to remote controlled electromechanical devices. More specifically, this invention describes the process through which an individual can set customized limits that override the control requests sent by the remote party and constrain the behavior of the remotely controlled electromechanical device(s), to ensure its behavior stays within certain parameters.

2. Prior Art

A number of systems and inventions have been developed that allow for a person to control an electromechanical device in a remote location.

Examples of remote controlled electromechanical devices include U.S. Pat. No. 6,368,268 entitled “Method and device for interactive virtual control of sexual aids using digital computer networks” to Sandvick et al. which allows the user to remotely control a sexual device over a computer network. Other examples of companies that offer products facilitating the remote control of a personal product or sexual devices include WebPower Inc. which markets and sells a device called “the SafeSexPlus adapter”, Feelthe.net LLC markets a product called “Sexsations” under the trade name DigitalIntamacy.com., and Cyber-Vibe. Lastly, Doc Johnson recently announced a version of the iVibe personal massager/vibrator that (through a license agreement with Hi Joy Products) can be controller by a remote party over the internet.

All of the aforementioned prior art examples allow a remote party or “operator” to control a sexual device in a remote location where a “recipient” receives some form of stimulation from said device. For clarity, the “operator” is defined as the person who is controlling an electromechanical device that exists in a remote location. The “recipient” is the person in physical possession of the electromechanical device being controlled by the “operator”.

All of the prior art examples place complete control of the device with the operator. The recipient (the person who is actually receiving stimulation from the device) does not have the capability to limit or constrain the acceptable behavior of the device. For example, if the device being controlled is intended to provide physical stimulation, the remote operator may unwittingly cause the device to behave in a manner that exceeds that which is preferred by the recipient.

Alternatively, if the device is a remote controlled car, the remote operator might cause the car to go faster than desired by the individual who is in physical possession of the car.

In all examples of prior art where a mechanical or electrical device being remotely controlled by an operator, it seems to be highly desirable for the recipient to be able to set, and change at will, limits or parameters that define the acceptable behavior of the device in question.

However, none of the prior art enables the recipient to put limits on the acceptable behavior of device being remotely controlled by the operator.

### OBJECT AND ADVANTAGES

The purpose of the invention is to provide an individual with the ability to customize and define parameters that restrict the behavior of an electromechanical device being remotely controlled by a remote party.

The invention described herein provides the ability for the recipient to define, and dynamically change, limits that govern the behavior of the device and that over ride the control requests sent by the operator. In this way, the recipient can rest assured that the device will not behave in a manner inconsistent with their wishes.

In the preferred embodiment of the invention, the recipient can dynamically change or alter the limits of the device—even during use.

In the preferred embodiment of the invention, the recipient will receive visual or auditory cues that indicate when a control request is made by the operator that exceeds the limits set by the operator. In this way, if desired, the operator will not know that the recipient has constrained the behavior of the device and will have the illusion of complete control.

In an alternate embodiment, the remote operator would also be provided with visual cues, auditory cues, or other stimulus that would alert said operator that their intended action exceeded the parameters set by the recipient.

This combination of the above objects and advantages represents a significant advantage over the prior art. The objects and advantages are not limited to those described above. Additional objects and advantages will become apparent from a consideration of the drawings and ensuing description.

### SUMMARY

The process provides for an individual to limit the behavior of an electromechanical device being controlled by a different person in a remote location.

The invention presented herein is superior to the prior art in that it:
Allows the recipient to set boundaries on acceptable use
Adds a level of safety for the recipient
Enables the recipient to change the behavioral limits of the electromechanical device—even while it is in use
Provides visual or auditory cues that allow the recipient to learn what action was desired by the operator
Can (if desired) maintain the illusion of control - allowing the operator to believe that she or he is in complete control of the electromechanical device and to remain unaware of limits established by the recipient
Can (if desired) educate the remote operator that he, she, or it has exceeded a desired parameter—allowing the operator to learn about the perceived limits desired by the recipient.

### DRAWINGS

FIG. 1 is a diagram that illustrates the relationship between the remote party or “operator” and the “recipient” in physical possession of a device being controlled by the operator.

FIG. 2 is a flow chart describing how the recipient defines limits for the device and how said limits can be changed during the interaction between operator and recipient.

FIG. 3 is a flow chart describing how control signals are generated by the operator, compared to the limit value, and either released or diminished and sent on to the electromechanical device. This figure illustrates how information is provided to the recipient about the nature of requests sent by the remote operator. This flow chart also includes an alternative embodiment in which the remote operator receives educational information about the limits established by the recipient including notification that a given request exceeded the value of the current limits established by the recipient.

### REFERENCE NUMERALS USED IN FIGURES

10 The Operator: the remote party in control of the device

20 Communication Network

30 The Recipient

40 An electromechanical device being used by the recipient, but that is controlled by the operator

50 The Recipient sets a limits that defined the acceptable behavior of the device

51 The device is controlled by the remote operator

52 The recipient, or the person physically interacting with the device, can evaluate if the limits established in step 50 are appropriate

53 The user may increase or decrease limits on the device while the remote operator continues to send requests and instructions to the device

60 [Start] The remote user generates a control request

61 Control request received

62 The recipient is provided with information about the nature of the control request

63 Control request is compared to limit value defined by recipient

64 In the event the value does not exceed the limit, the value is transmitted to the device

65 In the event the value exceeds the limit, the recipient is notified via visual or auditory information that describes the amount by which the requested value exceeds the limit

66 The device behaves consistent with the value provided in step 64 OR 65

67 The device continues operation until it receives the next command

70 In an alternate embodiment the remote operator may be notified a request has exceed a limit

71 To help educate the remote operator that their request has exceeded the limit established by the recipient, a visual cue, auditor cue, or other stimulus is provided to the operator.

72 No information about limits is provided to the operator and said operator maintains the illusion of controller over the device.

### DETAILED DESCRIPTION

Operation—Preferred Embodiment

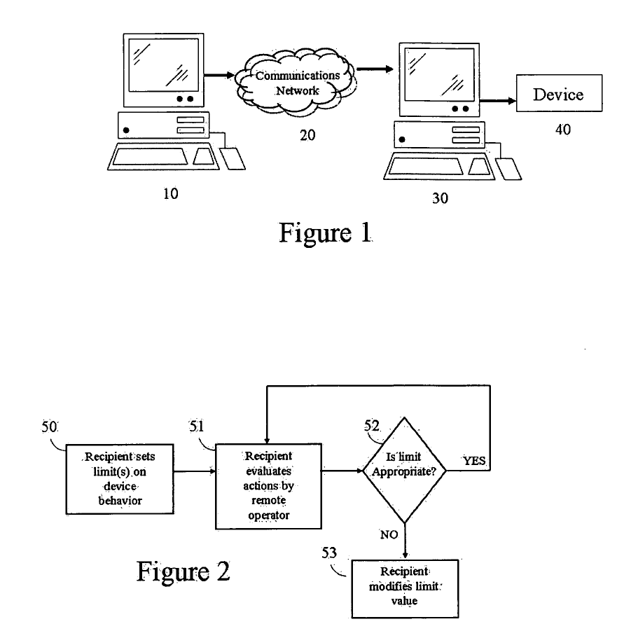

FIG. 1 illustrates the relationship between the Operator's computer 10, and the Recipient's computer 30. Control signals are generated on the operator's computer 10, and sent through a communications network 20, where they are received by the Recipient's computer 30. The Recipient's computer compares the control request with the limit(s) defined by the recipient and releases the appropriate control value(s) to device 40.

FIG. 2 is a flow chart describing how the recipient defines, evaluates, and (if appropriate) alters the limits defined for the device. In step 50 the recipient defines one, or more values that constrain the behavior of the device. In steps 51 and 52 the recipient evaluates the remote operator attempts to control device. If desired, as noted in step 53, the recipient can increase or decrease one or more of the values that constrain the device.

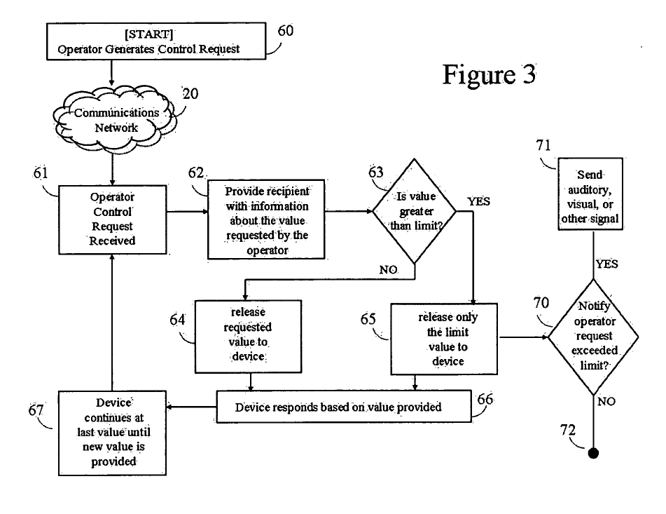

FIG. 3 is a process map describing in detail how the control requests sent by the remote operator are compared to limit values and how the resulting comparison is used to determine the behavior of the device. The remote operator generates a control request in step 60 and sends the request through communications network 20. The request is received in step 61. In step 62 the recipient is provided with real time information about the control requests. At step 63 the value of the control request is compared to the limit value(s) defined by the recipient. If the requested value(s) is less than or equal to the limit value then in step 64 the value is released to the device. If the requested value exceeds the limit value set by the recipient, then in step 65 the recipient is notified of the amount in excess of the limit value using visual or auditory cues and only the limit value is released to the device. In step 66 the device responds to the control value provided and at proceeds to step 67 where it will await the next control signal.

Operation—Additional Embodiment

FIG. 3 is also used to illustrate what information, if any, is provided to the remote operator about requests that exceed the limits. In step 71 the remote operator is provided with auditory alerts, visual cues, or other stimulus to notify the operator that a request exceeded the current limits. In Step 72 the remote operator is not provided with any information about the limits established by the recipient and will (if desired) be provided with the illusion of complete control over the device.

### CONCLUSION

Thus the reader will see that the invention described herein provides the valuable ability for an individual to establish and manage limits that govern the behavior of a device that is being controlled by a remote operator. The ability to create and manage limits that over ride the control requests sent by the operator ensure that the device does not exhibit behavior that is unsafe or unwise. In this way, the recipient can rest assured that the device will not behave in a manner inconsistent with their wishes. Ultimately, the limits create a safer environment and more comfortable environment for the recipient. Scope

While the above description contains many specifics, these should not be construed as limitations on the scope of the invention, but rather as an exemplification of one preferred embodiment thereof. For example, while prior art examples are limited to devices providing sexual stimulation, the invention is applicable to any situation in which an operator uses a communications network to partially or completely control an electrical or mechanical device. Examples of devices that may be controlled (and governed) in this manner include vehicles, children's toys, pet toys, home/kitchen appliances, lighting, systems, heat/air conditioning, irrigation/plumbing/water management systems, and/or industrial equipment. Accordingly, the scope of the invention should be determined not by the embodiments illustrated, but by the appended claims and their legal equivalents.

Appendix Item 1—Server Code

The example below is code that can be used to create an application that provides the limit feature described herein.

if (old_motor_a != last_motor_a ∥ old_motor_b !=
last_motor_b)

{

old_motor_a = last_motor_a;
old_motor_b = last_motor_b;
if (motor_a > sliderA−>getValue( ))
{

motor_a = sliderA−>getValue( );
progressA−>setRedline(motor_a);

}
progressA−>setProgress(last_motor_a);

if (motor_b > sliderB−>getValue( ))

{

motor_b = sliderB−>getValue( );
progressB−>setRedline(motor_b);

}
progressB−>setProgress(last_motor_b);

// send values to USB device

if (sin_write_usb(0, motor_a, motor_b) == −1)
{

noUSB( );

}

1. A process for an individual to limit the behavior of an electrical or mechanical device being controlled by a different person in a remote location comprising
(a) A method for allowing an individual to define behavioral limits
(b) A mechanism that will compare requests from remote parties with current limit values
(c) A means of releasing the lesser of the original requested value or the limit value to said device
whereby a person employing this method can limit the behavior of a device being controlled from a remote location.
